(19) United States
(12) Patent Application Publication
(10) Pub. No.: US 2009/0136929 A1
(43) Pub. Date: May 28, 2009

(54) NOVEL IN VITRO METHOD OF QUANTIFYING DEMINERALIZED BONE OSTEOINDUCTIVITY

(75) Inventors: Rama Akella, Austin, TX (US); Julie R. O'Connell, Lakeway, TX (US)

Correspondence Address: WOOD, HERRON & EVANS, LLP (ZIMMER), 2700 CAREW TOWER, 441 VINE STREET, CINCINNATI, OH, 45202, US

(73) Assignee: ZIMMER ORTHOBIOLOGICS, INC., Austin, TX (US)

(21) Appl. No.: 11/946,411

(22) Filed: Nov. 28, 2007

(51) Int. Cl. C12Q 1/68; C12Q 1/02

(52) U.S. Cl. 435/6; 435/29

(57) ABSTRACT

The present invention provides an in vitro method for determining the osteoinductive potential of a biomaterial. In particular, the method measures the expression of osterix by osteoblast progenitor cells incubated with the biomaterial. In various embodiments, the biomaterial is demineralized bone (DMB) and the progenitor cells are incubated with DMB or proteins extracted from DMB.

### TECHNICAL FIELD

The present invention relates generally to a method for determining the osteoinductivity of demineralized bone (DMB) and, more specifically, to determining osteoinductivity based on the ability of DMB to induce expression of osterix.

### BACKGROUND

Autologous cancellous bone (ACB) is generally considered the gold standard for bone grafts because it is osteoinductive, non-immunogenic and, by definition, has the appropriate structural and functional characteristics for a particular recipient. Unfortunately, ACB is only available in a limited number of circumstances. For example, some individuals lack ACB of appropriate dimensions and quality for transplantation. Moreover, donor site morbidity can pose serious problems for patients and their physicians. Thus, much effort has been invested in the identification or development of alternative bone graft materials.

Demineralized bone matrix (DBM) implants have been reported to be particularly useful. Demineralized bone is typically derived from cadavers. The bone is removed aseptically and/or treated to kill any infectious agents. The bone is then pulverized under controlled temperature to small particles by milling or grinding. The mineral component is then extracted (e.g., by soaking the bone in an acidic solution). The remaining matrix is malleable and can be further processed and/or formed and shaped for implantation into a particular site in the recipient. Demineralized bone prepared in this manner contains a variety of components including proteins, glycoproteins, growth factors, and proteoglycans.

DMB induces cellular recruitment to the site of implantation. The recruited cells may eventually differentiate into bone forming cells. Such recruitment of cells leads to an increase in the rate of wound healing and, therefore, to faster recovery for the patient. In addition to the active factors present within the DMB, the overall structure of the DMB implant is also believed to contribute to the bone healing capabilities of the implant. The osteoinductivity of demineralized bone matrix is highly variable and can be attributed to differences in age, lifestyle and gender of the donor, as well as variations in preparation, sterilization, and storage techniques. Osteoinduction is the generation of new bone-forming cells from non-differentiated cells. Different methods of processing generate DMB with dissimilar physical properties, including residual mineral and osteoinductive protein content, particle size and geometry, which also affect DMB osteoinductive potential. Therefore, each lot of DMB must be screened for adequate osteoinductivity prior to market release.

Methods currently utilized to measure DMB osteoinductivity rely on in vivo assessments using an athymic rat muscle pouch model. While this method has proven to be successful, the procedure is rather cumbersome, having a minimum 5-week turnaround time, and the objective quantification of osteoinductive potential is relatively difficult. In addition, animal experiments are costly and require the expertise of surgeons, animal care facility providers, and trained pathologists.

Currently practiced in vitro methods include co-culturing DMB or proteins released from DMB with osteoprogenitor cells or myoblasts to induce differentiation down the osteoblastic lineage. The acquisition of specific markers of mature osteoblasts, such as expression of alkaline phosphatase, either alone or in conjunction with additional specific markers, including osteopontin, osteocalcin, and bone sialoprotein, are the measures of osteoinductive potential. The disadvantage of this approach is that the identification and quantification of each of these proteins requires a separate assay and furthermore, a clear correlation between such in vitro methods and in vivo DMB osteoinductivity has yet to be firmly established. A second in vitro method uses either guanidine hydrochloride extraction or collagenase digestion to extract non-collagenous osteoinductive proteins from DMB and measures the concentrations of various bone morphogenetic proteins (BMPs) of the resulting extracts. This method relies on the assumption that DMB associated osteoinductive proteins (e.g., BMPs) are the primary determinant of DMB osteoinductivity and therefore, the assay focuses on quantification of these proteins. A further shortcoming of this methodology is the inability to determine if the BMPs thus measured are functionally active.

Thus, there is a need for a quantitative method of determining the osteoinductivity of DMB that is rapid and accurate and relatively inexpensive.

### SUMMARY OF THE INVENTION

One embodiment of the present invention is a method of measuring osteoinductivity of demineralized bone (DMB). The method includes culturing cells in contact with protein from the DMB to induce osteodifferentiation of the cells, followed by measuring the expression of osterix (Osx) in the cells.

Another embodiment is a method of measuring osteoinductivity of demineralized bone (DMB) including culturing progeniter cells with osteoblastic potential in the presence of DMB to induce osteodifferentiation of the cells. RNA is extracted from the cells and used to synthesize cDNA. The extent of osterix mRNA expression of the cDNA is determined using quantitative PCR (qPCR).

Another embodiment is a method of measuring osteoinductivity of demineralized bone (DMB) including extracting osteoinductive proteins from DMB and culturing progenitor cells with osteoblastic potential in contact with the extracted osteoinductive proteins to induce osteodifferentiation of the cells. RNA is extracted from the cells and used to synthesize cDNA. The extent of osterix mRNA expression of the cDNA is determined using qPCR.

In another embodiment, a kit is provided for measuring osteoinductivity of demineralized bone (DMB). The kit includes an osterix-specific PCR primer set with primers, an osteoinductive protein to be used in a control condition, and instructions for co-culturing the DMB proteins with osteoprogenitor cells and measuring osterix expression.

### BRIEF DESCRIPTION OF THE DRAWINGS

FIG. 1 shows DMB induced osterix (Osx) mRNA expression in various lots of DMB as determined by RT-qPCR according to one embodiment of the invention.

FIG. 2 shows DMB derived protein lysate induced osterix (Osx) mRNA expression in various lots of DMB as determined by RT-qPCR according to one embodiment of the invention.

FIG. 3A shows electrophoretic separation of the semi-quantitative RT-PCR products of Osx mRNA expression induced by different concentrations of DMB-protein from two different lots of DMB as indicated in one embodiment of the method.

FIG. 3B shows the qPCR results quantifying Osx mRNA expression from same samples as shown in FIG. 3A representing the correlation between semi-quantitative and qPCR.

### DETAILED DESCRIPTION

The present invention provides a method of measuring osteoinductivity of a synthetic or non-synthetic biomaterial. In one embodiment, the biomaterial is demineralized bone (DMB). The method involves quantifying the induction of a “master switch” transcription factor, osterix (Osx), by the biomaterial. Osx is an essential component driving osteoblast differentiation and is responsible for the expression of various osteoblast-specific markers including alkaline phosphatase, osteopontin, osteocalcin, and bone sialoprotein. Osx is a zinc finger-containing transcription factor that is specifically expressed in developing bones. One advantage of the method is the ability to effectively analyze the induction of a panel of osteoblast-specific markers in a single assay by quantifying the induction of the single protein responsible for initiating the transcription and expression of many osteoblast marker proteins. Furthermore, because of the central role Osx plays in directly orchestrating the induction of multiple osteoblastic signaling pathways, measuring its DMB-induced expression is likely to be a more accurate measure of DMB osteoinductivity than currently practiced in vitro methods.

The method uses DMB proteins to promote differentiation of progenitor cells, also referred to as assay cells, into osteoblasts (OB). In one embodiment, the osteoinductive measure is the induced mRNA expression levels of Osx in the assay cells.

In one embodiment, the method comprises culturing cells with proteins extracted from DMB to induce osteodifferentiation of the progenitor cells and the subsequent measurement of Osx expression of the cells. In one example, the protein extracted and/or released from DMB is non-collagenous protein. As shown in FIG. 2, Osx mRNA expression induced by DMB-protein, and thus the osteoinductivity of the DMB, is variable depending on the specific lot of DMB. The protein can be extracted one or more times from DMB by chemically treating the DMB. In one example, DMB is chemically treated with 4.0M guanidine hydrochloride for 48 hours with constant agitation at 150 RPM at 25° C. The extracted proteins in solution are separated from the DMB matrix by centrifugation at 15,180 rcf for 10 minutes at room temperature and collected. Fresh 4.0M guanidine hydrochloride is added to the once extracted DMB matrix and subject to a further extraction of 24 hours, as described above, and the protein solution collected by centrifugation and combined with the first extract. The resulting matrix is referred to as devitalized bone matrix (DVBM). Other chemicals that can be used for extraction of proteins from DMB, as known to one skilled in the art, include reagents such as 6M to 8M urea, calcium chloride, lithium chloride, or mixtures thereof. In another example, the protein is released from DMB by collagenase digestion of the DMB. With collagenase digestion, DMB can be hydrated in buffer for about two hours at about 37° C. followed by addition of collagenase. In one embodiment, collagenase is added to the DMB to achieve a final concentration of about 5 U/mL to 100 U/mL and allowed to incubate for about 6-48 hours. In one example, the final concentration is about 20 U/mL, which is allowed to incubate for about 24 hours.

Following chemical and/or enzymatic treatment of DMB, non-collagenous, osteoinductive proteins, such as various bone morphogenetic proteins (BMPs), are released and/or extracted from DMB. In various embodiments, the DMB extract is subjected to further processing steps, including concentration, dilution, removal of constituents, addition of components, fractionation, etc. In addition to treating the assay cells with the DMB extract, the assay cells may also be treated with a positive and negative control condition. The positive control condition can include treating the assay cells with any one of a variety of osteoinductive proteins, e.g., bone morphogenetic protein 2 (BMP2). The negative control condition can include an absence of osteoinductive agent, e.g. DMB or BMP2.

Cells used in the method, referred to as assay cells, can include progenitor cells with osteoblastic potential. For example, assay cells are capable of osteodifferentiation when cultured with the DMB extract. In one example, the assay cells are mesenchymal stromal cells (MSCs). In another example, the assay cells are W20-17 cells, which is a mouse bone marrow derived cell line. Other suitable cell lines known in the art include C2C12 cells, CH310t1/2cells, and the like. The assay cells can be plated in wells of a multi-well plate, e.g. 24-well, 48-well, 96-well, etc., for subsequent assay. The DMB extract is added to the assay cells and the mixture is incubated for a time ranging from about 8 hours to about 72 hours. In one embodiment, the assay cells are incubated with the DMB extract for about 48 hours.

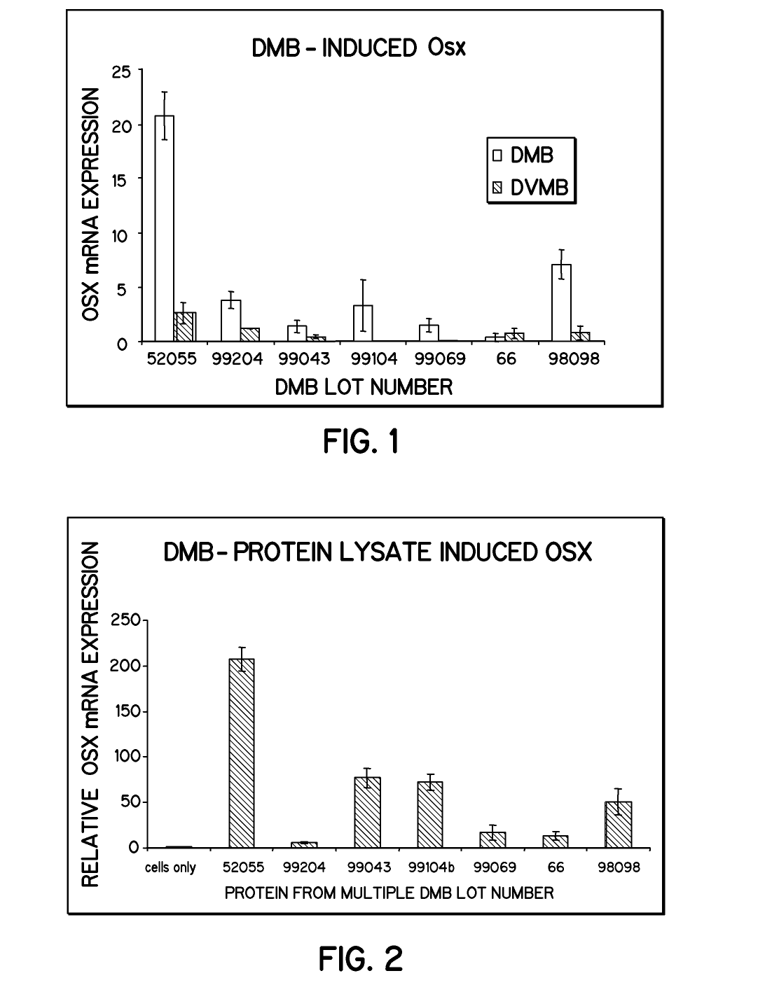

In another embodiment, the method includes culturing the assay cells with DMB to induce osteodifferentiation of the cells and the subsequent measurement of Osx expression of the cells. As shown in FIG. 1, Osx mRNA expression induced by DMB, and thus the osteoinductivity of the DMB, is variable depending on the specific lot of DMB. Typically, bone is ground to obtain particles in the size range of 100-1000 microns before demineralization to achieve optimal demineralization and exposure of osteoinductive proteins. Subsequently it is neutralized with buffered salt solutions and equilibrated with complete cell culture medium prior to co-culturing with the assay cells. The method utilizes direct contact between the cells and the DMB to stimulate osteodifferentiation of the assay cells and the induction of Osx expression. In one embodiment, non-tissue culture treated plates and/or wells are used to co-culture the assay cells and DMB to maximize the contact between the assay cells and the DMB. The non-tissue culture plates and/or wells help to ensure that the only available substrate for cell attachment is the DMB. Without being held to a single theory, it is believed that the present method quantifies the dual influence of both the DMB diffusible, osteoinductive proteins and the direct cellular contact with matrix-bound growth factors of the DMB on cellular induction. Thus, maximizing cell-to-DMB contact has the added advantage of more accurately mimicking the cell's natural, in vivo environment. Additionally, creating maximal contact between the assay cells and the DMB provides for the additional cellular response to the physical characteristics, e.g. shape, size and residual mineral content, of the DMB. Geometry and cell substrate relationships have been shown to be essential components in evaluating osteoinduction in vitro.

In one embodiment, the assay cells and the DMB are co-cultured for a time ranging from about 48 hours to about 168 hours. In one example, the assay cells and the DMB are co-cultured for about 72 hours. In addition to co-culturing the assay cells with DMB, the assay cells may also be treated with a positive and negative control condition. The positive control condition can include treating the assay cells with bone morphogenetic protein 2 (BMP2). The negative control condition can include an absence of osteoinductive agent, e.g. DMB or BMP2.

Following incubation of the assay cells with either the DMB extract or the DMB, the level of Osx expression is measured in the assay cells. In one embodiment, Osx expression is measured by determining the amount of Osx mRNA present. The amount of Osx mRNA can be determined relative to a control mRNA level. Examples of control mRNAs include internal housekeeping genes such as adenosine triphosphate synthase (ATPS). The control is used to correct for initial cDNA template quantity. In another embodiment, the control mRNA level represents Osx mRNA level in untreated cells such that Osx mRNA in assay cells following incubation with DMB or DMB-proteins is compared to the level of Osx mRNA in untreated cells. In another embodiment, Osx expression may be measured by determining the amount of Osx protein by using known techniques, such as monoclonal antibodies.

Following incubation with DMB or DMB extracts, the assay cells are lysed and the cellular RNA is isolated. RNA can be isolated using TRIZOL® reagent (See example below). In another embodiment, the use of filters having affinity for nucleic acids may be used, such as those manufactured by Ambion or Qiagen. The isolated RNA is then used to synthesize first strand complimentary DNA (cDNA) by reverse transcription using one of various commercial kits that are available and known to one skilled in the art.

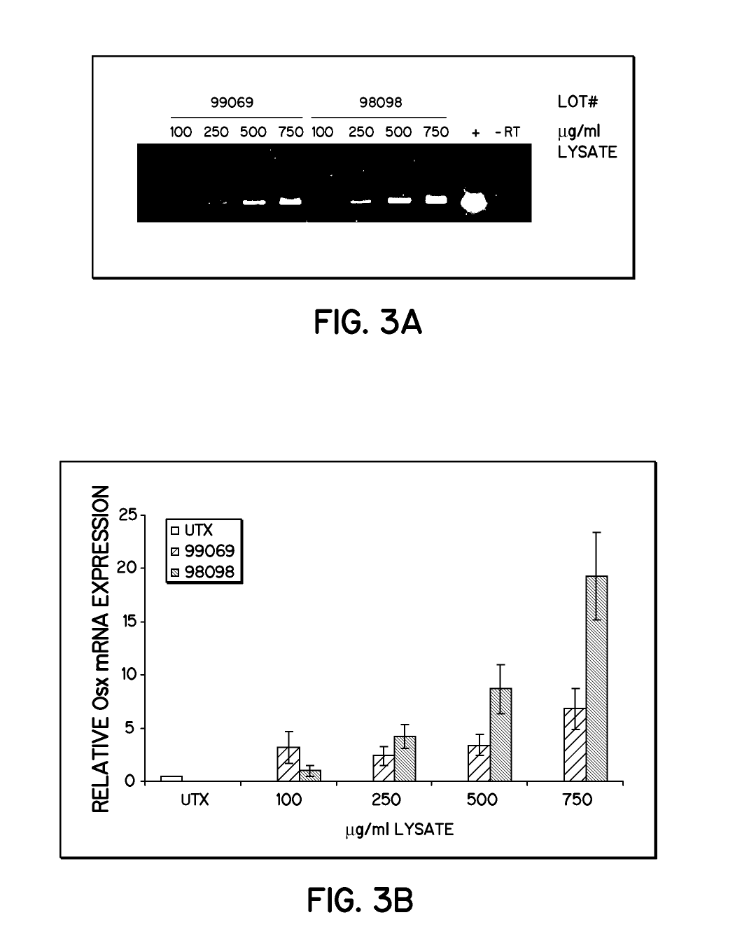

The relative amount of Osx cDNA is then determined using methods known to one skilled in the art, including polymerase chain reaction (PCR). In one embodiment, semi-quantitative RT-PCR is used to approximate the relative quantity of Osx cDNA in the sample. For instance, Osx mRNA is reverse transcribed into cDNA, which is then amplified by PCR, with the resultant product being subjected to agarose gel electrophoresis. With reference to FIG. 3A, the relative intensity of the product bands is compared from various conditions. The various conditions can include different lots of DMB and control conditions. The primers used for RT-PCR of the Osx mRNA may include sequence 5′ TTCTAGTCAAATGCATCTCTGTAT 3′ (SEQ ID NO: 1) and sequence 5′ ACCTCCAAACCAAAATCC TCCTGT 3′ (SEQ ID NO: 2), which results in a product of about 401 base pairs (bp).

With reference to FIG. 3B, the relative amount of Osx cDNA may be determined by quantitative PCR (qPCR). qPCR is a modification of the polymerase chain reaction and is used to rapidly measure the quantity of DNA, complementary DNA or ribonucleic acid present in a sample. Like other forms of polymerase chain reaction, DNA samples are amplified using temperature-dependent/sensitive DNA polymerases. In general, the amount of DNA is measured after each cycle of PCR by the inclusion of fluorescent markers in the reaction mixture. In one embodiment, Osx is quantified using the taqman FAM-dye labeled probe with primers for Osx (Applied Biosystems). The quantitation of ATP synthase may be used to control for variations in total mRNA (Applied Biosystems).

In addition, two separate normalizations can be used in the qPCR analysis method with the data being normalized for i) variations in quantity of the starting cDNA template and ii) to a calibrator, such as gene expression by cells treated with a positive control like BMP2. Here, all data is expressed as % of the calibrator condition. In one embodiment, analysis of qPCR quantification is determined using the comparative CT (cycle threshold) calculation method with the normalizer being ATPS and the calibrator being an internal positive control, such as rh-BMP2 treatment. The comparative CT method of relative qPCR quantification requires that the data be expressed relative to a standardized control, such as a calibrator.

A kit is also provided for measuring osteoinductivity of demineralized bone (DMB). In one embodiment, the kit includes an osterix-specific PCR primer set, an osteoinductive protein, e.g., BMP, to be used in a control condition, and instructions for co-culturing the DMB proteins with osteoprogenitor cells and measuring osterix expression. In one example, the osterix-specific PCR primer set includes primers having the sequence 5′ TTCTAGTCAAATGCATCTCTGTAT 3′ (SEQ ID NO: 1) and sequence 5′ ACCTCCAAACCAAAATCC TCCTGT 3′ (SEQ ID NO: 2). The kit can further include a chemical and/or enzymatic DMB-protein extracting reagent. In one example, the chemical DMB-protein extracting reagent is guanidine hydrochloride. In another example, the enzymatic DMB-protein extracting reagent is collagenase.

The following example further illustrates embodiments of the method.

### EXAMPLE

### DMB Induction of Osx:

Note: All steps are preferably carried out under aseptic conditions.

0.1 g of each DMB to be analyzed was placed in a single well of a non-tissue culture treated 24-well plate. 1 ml complete medium (DMEM containing 10% FBS and 1x Penn/Strep from Gibco) was added to each well and mixed on an orbital shaker for two minutes then transferred to a 37° C. tissue culture incubator for 30 minutes. The media was removed and the DMB was washed an additional three times. The final wash was left on the DMB for 18-24 hours. Then, the media was removed and 500,000 W20-17 cells were plated per well in 1 ml complete medium and 500,000 cells were plated in each of 2 blank wells for positive and negative controls. 600 ng of rhBMP2 was added to the positive control well and the plate was incubated for 48 hours at 37° C. in an atmosphere containing 5% carbon dioxide and 95% relative humidity. The medium was then removed from each well and replaced with fresh complete medium. The positive control was given fresh complete medium containing 600 ng BMP2, and the plate was incubated for an additional 24 hours at 37° C. as described above. The medium was then replaced with 800 μl Trizol® Reagent, followed by a brief vortex to mix the contents in the wells. The DMB particles and the Trizol® Reagent were then transferred to a microcentrifuge tube using a Pipetman® P1000.

### Procedure for RNA Isolation Using Trizol Reagent:

All tubes were vigorously vortexed for about one minute and then allowed to sit at room temperature (RT) for five minutes. The samples were then centrifuged at 12,000×g for 10 min at 4° C. and the supernatant was transferred to a new tube. 0.2 ml of chloroform was added to each sample (per 0.8 ml of Trizol) and shaken vigorously by hand for 15 seconds and then maintained at RT for 2-3 minutes. The samples were centrifuged at 12,000×g for 15 minutes at 4° C. The aqueous phase was transferred to a new tube and 0.01-0.02 volumes (based on volume of aqueous solution) of linearized acrylamide were added to each tube as an RNA carrier, and mixed by vortex. Isopropyl alcohol (0.5 mL) was added to each sample to precipitate RNA, mixed briefly by vortex, and incubated at RT for 10 minutes. The samples were then centrifuged at 12,000×g for 10 minutes at 4° C. The isopropanol was decanted and the RNA was seen as a clear pellet at the bottom of tube. The pellet was washed with 1 ml of 75% ethanol and then vortexed and spun at no more than 7500×g for five minutes. The RNA can be stored in 75% ethanol at −20° C. for about a year. For subsequent use, the pellet was resuspended in 30 μl Elution solution from the RNAqueous-4PCR kit (Ambion) that was heated to 60° C. The pellet was resuspended by pipetting up and down and DNase was added to degrade genomic DNA. The RNA concentration was quantified using Nanodrop® by measuring absorbance at 260 nm. Depending on the absorption wavelength, the Nanodrop® method can be used to quantitate both proteins as well as nucleic acids, with absorption at 260 nm measuring nucleic acid concentration, and absorption at 280 nm measuring protein concentration.

Protein Extraction from DMB:

DMB is chemically treated with 4.0M guanidine hydrochloride—Tris-HCl (pH7.4) (0.2 gDMB/mL of reagent) for 48 hours with constant agitation at 150 RPM at 25° C. The extracted proteins in solution are separated from the DMB matrix by centrifugation at 6441 g, room temperature, for 10 minutes and collected. Fresh 4.0M guanidine hydrochloride—Tris-HCl (pH7.4) (same volume as in first extraction) is added to the once extracted DMB matrix and subject to a further extraction of 24 hours as described above and the protein solution collected by centrifugation and combined with the first extract. The pooled extracts are subject to high speed centrifugation at 15,180 RCF for 10 min at room temperature to remove all visible particulate material.

DMB (1 g) was treated with 20 u of collagenase in the presence of 6.5 ml collagenase buffer containing 0.2 M Tris-HCl buffer (pH7.2), 20 mM NaCl₂, 3 mM CaCl₂, 3 mM MgSO₄, 3 mM NEM (N-ethylmaleimide), 0.1 mM phenylmethanesulfonyl fluoride (PMSF), and 0.1 mM benzamidine-HCl, and incubated at 37° C. for 24 hours with shaking. The mixture was then centrifuged at 6000×g for 10 minutes and the supernatant was collected. The resulting protein concentration was quantitated. The extract can be frozen at −80° C. for later use. W20-17 cells were plated at 100,000 cells/well in complete medium on 24-well tissue culture treated plates. The plate was placed in a cell culture incubator for 1 to 1.5 hours to allow cells to attach to the wells. The media was then aspirated from the wells and 1 ml of complete medium containing 750 μg/ml DMB extracted proteins was added to the wells. The negative control condition only used 1 ml of complete medium and the positive control condition had 1 ml of complete medium with 600 ng/ml BMP2. The tissue culture plates were placed in a tissue culture incubator for 48 hours. RNA was extracted from the cells.

RNA Isolation from Cells Treated with DMB Protein Extract:

RNA isolation was performed using the RNAqueous-4PCR kit (Ambion) according to the manufacturers' instructions. Briefly, the tissue culture wells containing the cells were rinsed with phosphate-buffered saline (PBS) and then incubated with 350 μl RNA lysis buffer per well. The wells were then scraped with a cell scraper to ensure cell detachment. The resulting cell lysate/suspension was transferred to a 1.5 ml RNase-free eppendorf tube and vortexed vigorously to lyse any remaining intact cells. 350 μl of ethanol (supplied in the kit) was added to the lysate and mixed gently by inverting the tube several times. The lysate/ethanol mix was applied to a filter cartridge (in kit) assembled in a collection tube. The filter assembly was centrifuged at 15,000×g for 30 sec to pull solution through filter. The flow-through was discarded and the filter was washed three times with the wash solution. After the last wash was discarded, the RNA was eluted from the filter by adding 40 μl of elution solution at 40-60° C. directly on to the center of the filter and centrifuging the filter at 15,000×g for 30 seconds. The filter was eluted a second time by adding an additional 20 μl of the same elution solution to the filter and centrifuging as above. The samples were treated with DNase I to digest any genomic DNA followed by subsequent addition of DNase I inactivation solution. The DNase I slurry was pelleted by centrifugation and the resultant RNA solution was removed, quantitated and either used for cDNA synthesis or stored at −80° C.

cDNA Synthesis:

cDNA is synthesized using available commercial kits such as Invitrogen SuperScript® First-Strand Synthesis System for RT-PCR. Other commercially available kits may be used for cDNA synthesis steps.

Quantify cDNA Concentration

cDNA concentration was determined using standard spectrophotomeric techniques known to one skilled in the art.

### Semi-Quantitative RT-PCR Using Custom Designed Mouse Osterix Primers:

PCR was performed on the synthesized cDNA using Osterix-specific primers SEQ ID NO: 1 and SEQ ID NO: 2, which results in a product size of 401 bp. The samples were heat denatured at 94° C. for 5 minutes prior to initiation of the PCR cycle which consisted of the following steps: 94° C. for 30 seconds, 53° C. for 30 seconds, and 72° C. for 30 seconds. The PCR cycle was repeated 25 times. The resultant RT-PCR products were electrophoresed to confirm the presence of a single amplified product of the expected size (401 bp).

qPCR to Quantify Osterix mRNA Expression Levels Using the Relative CT (Cycle Threshold) Method of Analysis:

qPCR was performed using an Applied Biosystems Inc. instrument according to the manufacturers' instructions. The Osx mRNA was quantified using the taqman® FAM-dye labeled probe with custom primers for Osx and ATPS. qPCR quantification was analyzed using Relative Quantification relying on the comparative CT method. The experimental data was normalized to the ATPS data, and the calibrator was an internal positive control (rh-BMP2, R&D systems, dosed at 600 ng/ml). The comparative CT method of relative qPCR quantification results in data that is expressed relative to a standardized control (calibrator above). In each Osx analysis, one well of the plate is treated with a positive control and the data is expressed relative to this standard.

It should be understood that the embodiments and examples described are only illustrative and are not limiting in any way. Therefore, various changes, modifications or alterations to these embodiments may be made or resorted to without departing from the spirit of the invention and the scope of the following claims.

What is claimed is:

1. A method of measuring osteoinductivity of demineralized bone (DMB) comprising:
culturing cells in contact with protein from the DMB to induce osteodifferentiation of the cells; and
measuring osterix expression of the cells.

2. The method claimed in claim 1 further comprising extracting RNA from the cultured cells and synthesizing cDNA from the extracted RNA; and
measuring osterix mRNA expression by the cDNA.

3. The method claimed in claim 2 wherein the cells are cultured in the presence of DMB.

4. The method claimed in claim 2 further comprising extracting protein from DMB and culturing the cells in the presence of the protein.

5. The method claimed in claim 2 wherein the cells are progenitor cells with osteoblastic potential.

6. The method claimed in claim 5 wherein the cells are stromal cells.

7. The method claimed in claim 4 wherein the protein is extracted by enzymatic treatment, chemical treatment, or combinations thereof.

8. The method claimed in claim 7 wherein the enzymatic treatment comprises treating the DMB with collagenase to extract non-collagenous protein from the DMB.

9. The method claimed in claim 7 wherein the chemical treatment comprises treating the DMB with guanidine hydrochloride to extract non-collagenous protein from the DMB.

10. The method claimed in claim 2 wherein the osterix mRNA is measured by qPCR.

11. A method of measuring osteoinductivity of demineralized bone (DMB) comprising:
culturing progenitor cells with osteoblastic potential in the presence of DMB to induce osteodifferentiation of the cells;
extracting RNA from the cells and synthesizing cDNA from the RNA; and
measuring osterix mRNA expression by the cDNA using qPCR.

12. The method claimed in claim 11 further comprising extracting protein from DMB and culturing the progenitor cells with osteoblastic potential in the presence of the protein to induce osteodifferentiation of the cells.

13. The method claimed in claim 12 wherein the protein is extracted by enzymatic treatment, chemical treatment, or combinations thereof.

14. The method claimed in claim 13 wherein the chemical treatment comprises treating the DMB with guanidine hydrochloride to extract non-collagenous protein from the DMB.

15. The method claimed in claim 13 wherein the chemical treatment comprises treating the DMB with guanidine hydrochloride to extract non-collagenous protein from the DMB.

16. The method claimed in claim 11 further comprising comparing osterix mRNA expression to a control mRNA level.

17. A method of measuring osteoinductivity of demineralized bone (DMB) comprising:
extracting osteoinductive proteins from the DMB;
culturing progenitor cells with osteoblastic potential in contact with the osteoinductive proteins to induce osteodifferentiation of the cells;
extracting RNA from the cells and synthesizing cDNA from the RNA; and
measuring osterix mRNA expression by the cDNA using qPCR.

18. The method claimed in claim 17 wherein the protein is extracted by enzymatic treatment, chemical treatment, or combinations thereof.

19. The method claimed in claim 18 wherein the chemical treatment comprises treating the DMB with guanidine hydrochloride to extract non-collagenous protein from the DMB.

20. The method claimed in claim 18 wherein the chemical treatment comprises treating the DMB with guanidine hydrochloride to extract non-collagenous protein from the DMB.

21. The method claimed in claim 17 further comprising comparing osterix mRNA expression to a control mRNA level.

22. A kit comprising
an osterix-specific polymerase chain reaction (PCR) primer set,
an osteoinductive protein to be used in a control condition, and
instructions for co-culturing the DMB proteins with osteoprogenitor cells and measuring osterix expression.

23. The kit of claim 22 further comprising a chemical and/or enzymatic DMB-protein extracting reagent.

24. The kit of claim 23 wherein the chemical DMB-protein extracting reagent is guanidine hydrochloride.

25. The kit of claim 23 wherein the enzymatic DMB-protein extracting reagent is collagenase.

26. The kit of claim 23 wherein primer set includes primers having sequence 5′ TTCTAGTCAAATGCATCTCTGTAT 3′ (SEQ ID NO: 1) and sequence 5′ ACCTCCAAACCAAAATCC TCCTGT 3′ (SEQ ID NO: 2).

27. The kit of claim 23 wherein the osteoinductive protein is BMP.


SEQUENCE LISTING (2 sequences)

SEQ ID NO: 1 - DNA, 24 bp, Homo sapien
ttctagtcaaatgcatctctgtat

SEQ ID NO: 2 - DNA, 24 bp, Homo sapiens
acctccaaaccaaaatcctcctgt